(19) United States
(12) Patent Application Publication
(10) Pub. No.: US 2005/0009027 A1
(43) Pub. Date: Jan. 13, 2005

(54) Reproduction of ribonucleic acids

(76) Inventors: Guido Krupp, Gnutz (DE); Peter Scheinert, Hamburg (DE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/488,231

(22) PCT Filed: Aug. 21, 2002
(86) PCT No.: PCT/EP02/09348

(30) Foreign Application Priority Data
Sep. 3, 2001 (DE) 10143106.6

(51) Int. Cl. C12Q 1/68; C12P 19/34

(52) U.S. Cl. 435/6; 435/91.2

(57) ABSTRACT

The present application relates to processes resulting in the amplification of ribonucleic acids. The processes comprise the following steps: (a) using a single stranded primer, an RNA-dependent DNA polymerase and deoxyribonucleotide monomers to synthesize a single stranded DNA via reverse transcription of RNA; (b) removing of the RNA; (c) using a single stranded primer comprising a promoter sequence, a DNA polymerase and deoxyribonucleotide monomers to synthesize a double stranded DNA; (d) separating the double stranded DNA into single stranded DNAs; (e) using a single stranded primer comprising a promoter sequence, a DNA polymerase and deoxyribonucleotide monomers to synthesize double stranded DNA on the basis of the single stranded DNA obtained in (d); (f) using an RNA polymerase and ribonucleotide monomers to synthesize multiple single stranded RNAs.

### CROSS-REFERENCE TO RELATED APPLICATIONS

This application is a national phase application under 35 U.S.C. § 371 of International Application Number PCT/EP02/09348, filed Aug. 21, 2002, the disclosure of which is hereby incorporated by reference in its entirety, and claims the benefit of German Patent Application Number 101 43 106.6, filed Sep. 3, 2001.

### INCORPORATION OF SEQUENCE LISTING

A paper copy of the Sequence Listing and a computer readable form of the sequence listing on diskette, containing the filed named “SeqList19006003.txt”, which is 489 bytes in size (measured in MS-DOS), and which was recorded on Mar. 2, 2004, are herein incorporated by reference.

### BACKGROUND OF THE INVENTION

To date, a multitude of processes resulting in the amplification of nucleic acids are known. The best known example is the polymerase chain reaction (PCR), developed by Kary Mullis in the mid-eighties (see, Saiki et al., Science, Vol. 230 (1985), 1350-1354; and EP 201 184).

In the PCR reaction single stranded primers (oligonucleotides with a chain-length of usually 12 to 24 nucleotides) anneal to a complementary, single stranded DNA sequence. These primers are subsequently elongated in the presence of a DNA-polymerase and deoxyribonucleoside triphosphates (dNTPs, namely dATP, dCTP, dGTP and dTTP) to obtain double stranded DNA. The double stranded DNA is separated by heating into single strands.

The temperature is reduced sufficiently to allow a new step of primer annealing. The primers are again elongated to double stranded DNA.

Repetition of the steps described above enables exponential amplification of the starting DNA, as the reaction conditions are adjusted such that almost each molecule of single stranded DNA will be transformed into a double stranded DNA within each round of amplification, melted into two single stranded DNAs which will be used again as templates for the next round of amplification.

If a reverse transcription reaction prior to the above process is carried out, wherein mRNA is transformed into single stranded DNA (cDNA) in the presence of an RNA-dependent DNA polymerase, a PCR reaction can be used directly for amplifying nucleic acids starting with an RNA sequence (see, EP 201 184).

A multitude of alternatives have been developed in the last years on the basis of this model reaction, which all differ with respect to the starting material (RNA, DNA, single or double stranded) and the reaction product (amplification of specific RNA or DNA sequences in a probe or the amplification of all sequences).

Over the last years, so called microarrays are used with increasing frequency for nucleic acids analysis. The essential component of such a microarray is a carrier plate onto which a multitude of different nucleic acid sequences (mostly DNA) were bound in different areas of the carrier. Usually, within one particular very area sector, only DNA with one specific sequence is bound, wherein one microarray may contain several thousand different areas which bind different sequences.

If these microarray are contacted with a number of nucleic acid sequences (mostly also DNA) obtained from a sample of interest under suitable conditions (salt content, temperature, etc.) complementary hybrids of nucleic acid sequences originating form the sample of interest and those bound to the plate are formed. Non-complementary sequences can be washed off. The areas on the microarray containing double stranded DNA can be detected and thus allow to conclude the sequence and the amount of the nucleic acid in the starting sample.

Microarrays are used to analyze expression profiles of cells, hence allowing the analysis of all mRNA sequences present in certain cells (see, Lockhart et al. Nat. Biotechnol. 14 (1996), 1675-1680).

Since the amount of mRNA available for this analysis is usually limited, special processes have been developed to amplify ribonucleic acids, which are to be analyzed by means of microarrays. For this purpose ribonucleic acids are optionally converted into the more stable cDNA form by means of reverse transcription.

Methods, yielding large amounts of amplified RNA populations of single cells are described in e.g., U.S. Pat. No. 5,514,545. This method uses a primer containing an oligo-dT-sequence and a T7-promoter region. The oligo-dT-sequence binds to the 3′-poly-A-sequence of the mRNA initiating reverse transcription of the mRNA. Subsequent to alkaline denaturation of the RNA/DNA heteroduplex a second DNA strand is prepared using the hairpin structure at the 3′-end of the cDNA as a primer and a linear double stranded DNA is obtained by opening via nuclease S1. The linear double stranded DNA is then used as template for T7 RNA polymerase. The resulting RNA can be used again as template for the synthesis of cDNA. For this reaction oligonucleotide hexamers of random sequences (random primers) are used. Following heat-induced denaturation, the second DNA strand is produced by means of the above-mentioned T7-olido-dT-primer and the resulting DNA can again be used again as template for T7 RNA-polymerase.

An alternative strategy is presented in U.S. Pat. No. 5,545,522, wherein a single oligonucleotide primer is used to yield high amplifications. RNA is reverse transcribed into cDNA using a primer having the following characteristics: a) 5′-dN₂₀, which means a randomly chosen sequence of 20 nucleotides; b) a minimal T7-promoter; c) GGGCG as transcription-initiation sequence; and d) oligo-dT₁₅. Synthesis of the second DNA strand is achieved by partial RNA digestion using RNase H, whereby the remaining RNA-oligonucleotides are used as primers for polymerase I. The ends of the resulting DNA are blunted by T4-DNA polymerase.

A similar process is disclosed in U.S. Pat. No. 5,932,451, wherein two so-called box-primers are further added to the 5′ terminal area, which enables double immobilisation by using biotin-box-primers.

However, the above mentioned processes to amplify ribonucleic acids have major disadvantages. All of the above mentioned procedures result in RNA populations which are different from the RNA populations present in the starting material. This is due to the use of the T7-promoter-oligo-dT-primers that primarily amplify RNA sequences of the 3′-section of the mRNA. Further, it has been shown that extremely long primers (more than 60 nucleotides) are prone to primer-primer-hybrids and thus also result in non-specific amplification of the primers (Baugh et al., Nucleic Acids Res., 29 (2001) E29). The known procedures therefore result in the production of a multitude of artefacts, interfering with the further analysis of the nucleic acids.

A problem underlying the present invention thus resides in finding a method to amplify ribonucleic acids, which allows homogeneous amplification of the ribonucleic acids present in the starting material. This problem is now solved by a method comprising the following main steps:
(a) using a single stranded primer, an RNA-dependent DNA polymerase and deoxyribonucleotide monomers to synthesize a single stranded DNA via reverse transcription of RNA;
(b) removing of the RNA;
(c) using a single stranded primer comprising a promoter sequence, a DNA polymerase and deoxyribonucleotide monomers to synthesize a double stranded DNA;
(d) separating the double stranded DNA into single stranded DNAs;
(e) using a single stranded primer comprising a promoter sequence, a DNA polymerase and deoxyribonucleotide monomers to synthesize double-stranded DNA on the basis of the single stranded DNA obtained in (d);
(f) using an RNA polymerase and ribonucleotide monomers to synthesize multiple single stranded RNAs.

### BRIEF SUMMARY OF THE INVENTION

The present invention provides a process for the amplification of ribonucleic acids comprising the following steps: (a) using a single stranded primer, an RNA-dependent DNA polymerase and deoxyribonucleotide monomers to synthesize a single stranded DNA via reverse transcription of RNA; (b) removing of the RNA; (c) using a single stranded primer comprising a promoter sequence, a DNA polymerase and deoxyribonucleotide monomers to synthesize a double stranded DNA; (d) separating the double stranded DNA into single stranded DNAs; (e) using a single stranded primer comprising a promoter sequence, a DNA polymerase and deoxyribonucleotide monomers to synthesize double stranded DNA on the basis of the single stranded DNA obtained in (d); and (f) using an RNA polymerase and ribonucleotide monomers to synthesize multiple single stranded RNAs. The present invention further provides kits which comprise the components required for performing the processes of the present invention.

The present invention also provides a process where the single stranded RNA has the same sense orientation (sequence) as the RNA starting material. The present invention also provides a process where the single stranded primer used in step (a) comprises an oligo-dT-sequence. The present invention also provides a process where in step (a) a 5′-(dT)₁₈V-primer is used for reverse transcription, with V being any deoxyribonucleotide-monomer different from dT. The present invention also provides a process where the RNA is hydrolysed using RNase in step (b). The present invention also provides a process where the RNA is removed using RNase I and/or RNase H in step (b). The present invention also provides a process where a single stranded primer is used in step (c) which comprises the sequence of a T7-, T3- or SP6-RNA-polymerase promoter. The present invention also provides a process where a single stranded primer is used in step (c) which comprises in addition to a promoter sequence a random sequence of not more than 6 nucleotides. The present invention also provides a process where a single stranded primer is used in step (c) with a total length of not more than 35, preferably not more than 30 nucleotides. The present invention also provides a process where a single stranded primer is used in step (c) having the sequence shown in SEQ ID NO: 1.

The present invention also provides a process where the Klenow-fragment of the DNA polymerase is used as DNA polymerase. The present invention also provides a process where the Klenow-exo⁻ DNA polymerase is used as DNA polymerase. The present invention also provides a process where dATP, dCTP, dGTP and dTTP are used as deoxyribonucleotide monomers. The present invention also provides a process where DNA double strands are separated in step (d) into single strands by means of heat. The present invention also provides a process where the single stranded primer in step (e) is identical with the single stranded primer used in step (c). The present invention also provides a process where the single stranded primer in step (e) is different from the single stranded primer used in step (c). The present invention also provides a process where T7-RNA polymerase is used in step (f) as RNA-polymerase. The present invention also provides a process where excess primer and/or primer-induced artefacts are removed prior to incubation with T7-RNA-polymerase. The present invention also provides a process where ATP, CTP, GTP and UTP are used as ribonucleotide monomers.

The present invention also provides a process where the amplification factor of the starting RNA sequence is at least 500, preferably at least 3000.

The present invention also provides a process wherein in step (a) a 5′-(dT)₁₈V-primer is used for reverse transcription; and in (b) an RNase is used to digest the DNA-RNA-hybrids; and in (c) and (e) a primer having the SEQ ID NO: 1 and the Klenow-exo DNA polymerase are used; and in (c) strand separation of double stranded nucleic acids is obtained using heat; and in (d) excess primers and/or primer-induced artefacts are first removed and then T7-RNA-polymerase is used.

The present invention also provides a process where the DNA double strands prepared in step (e) are separated into single strands and complementary DNA strands to each single strand are prepared using at least one single stranded primer, a DNA polymerase and the deoxyribonucleotide monomers. The present invention also provides a process where strand separation, primer annealing and elongation are repeated at least once, preferably at least 2 or 5 times. The present invention also provides a process where strand separation is achieved by means of heat.

The present invention also provides a process where further DNA double strands are produced using single stranded primers with the same sequence as the primer used in (c) and/or (e) The present invention also provides a process where a single stranded primer is used having the sequence shown in SEQ ID NO: 1. The present invention also provides a process where ribonucleic acids are produced with the same sequence orientation as the starting material and simultaneously also with the complementary sequence orientation.

The present invention also provides a kit for the amplification of ribonucleic acids, which comprises the following components: at least one single stranded primer comprising a promoter sequence; an RNA-dependent DNA polymerase; deoxyribonucleotide monomers; a DNA-dependent DNA polymerase; an RNA polymerase; and ribonucleotide monomers. The present invention also provides a kit further comprising: a 5′-(dT)₁₈V-primer for reverse transcription; RNase; a primer having the sequence shown in SEQ ID NO: 1; Klenow-exo⁻ DNA-polymerase; T7-RNA polymerase.

The present invention also provides a kit where the kit comprises two different single stranded primers. The present invention also provides a kit where one single stranded primer comprises an oligo-dT-sequence. The present invention also provides a kit where a single stranded primer comprises a 5′-(dT)₁₈V-primer sequence for reverse transcription, with V being any deoxyribonucleotide monomer different from dT. The present invention also provides a kit further comprising RNase I and/or RNase H. The present invention also provides a kit comprising a single stranded primer with a T7, T3 or SP6 RNA-polymerase promoter sequence. The present invention also provides a kit where wherein a single stranded primer comprises a promoter and further a random sequence of not more than 6 nucleotides.

The present invention also provides a kit comprising a single stranded primer having the sequence shown in SEQ ID NO: 1. The present invention also provides a kit comprising the Klenow-fragment of the DNA polymerase. The present invention also provides a kit comprising the Klenow-exo DNA polymerase. The present invention also provides a kit comprising the T7-RNA polymerase.

The present invention also provides a kit comprising a set of reagents for labelling and detection of nucleic acids. The present invention further comprises a kit comprising a microarray. The present invention further comprises a kit comprising instructions.

The present invention also provides a process for the analysis of nucleic acids, where ribonucleic acids are obtained, amplified using a process of the present invention and analyzed using a microarray. The present invention also provides a process for the analysis of nucleic acids, where the ribonucleic acids are obtained from a biological sample. The present invention also provides a process where the ribonucleic acids are amplified, converted to cDNA by means of reverse transcription, and the cDNAs are analyzed by using micoarrays. The present invention also provides a process where the amount and/or sequence of the cDNA are analyzed.

The present invention also provides a primer having the sequence of SEQ ID NO: 1.

### BRIEF DESCRIPTION OF THE DRAWINGS

FIG. 1 sets forth a schematic diagram of the process of the present invention.

FIG. 2 sets forth an alternate embodiment of the present invention. This alternative procedure includes amplification of the double stranded DNA, by means of PCR, prior to the transcription reaction.

### DESCRIPTION OF THE NUCLEIC ACID SEQUENCES

SEQ ID NO: 1 sets forth a nucleic acid sequence of a primer comprising a sequence of the T7-RNA-polymerase promoter.

### DETAILED DESCRIPTION OF THE INVENTION

Surprisingly, the process of the present invention leads to a homogeneous amplification of the ribonucleic acids present in the starting material. At the same time the process according to the invention prevents the production of artefacts. Hence the process according to the invention provides a significant improvement of methods to amplify ribonucleic acids and allows at the same time the improvement of procedures to analyze ribonucleic acids by means of microarrays.

One embodiment of the process according to the invention results in the amplification of a single stranded RNA with the same sequence (sense RNA) as the starting RNA. As an alternative embodiment it is possible to use the process according to the invention such that single stranded RNA of both orientations (same as starting RNA and the complementary sequence) can be obtained.

The single-stranded primer used in (a) preferably comprises an oligo-dT-sequence, a sequence containing several dT-nucleotides, with the advantage of primer binding to the poly A-tail of the mRNA. Hence resulting in reverse transcription of almost exclusively mRNA only.

In the process according to the invention it is preferred that the primer described in (a) comprises a 5′-(dT)₁₈V sequence. This refers to a primer having 18 dT-deoxyribonucleotide monomers followed by a single deoxyribonucleotide of different nature (namely dA, dC, or dG, here referred to as V). This primer almost exclusively allows reverse transcription of sequences which are located in the close vicinity of the 5′-end of the polyA-tail. The use of such a primer therefore suppresses the production of artefacts resulting from binding of the previously knwon oligo-dT-primers to larger polyA-areas in the mRNA.

Further, in the process according to the invention it is preferred that the RNA in the DNA-RNA-hybrids of (b) are digested by RNase. For this procedure any RNase can be used. The use of RNase I and/or RNase H is preferred. This step results in the elimination of all RNAs which have not been transcribed into cDNA during the first step of the procedure, particularly ribosomal RNAs, but also all other cellular RNAs which do not have the polyA-tail, characteristic for mRNAs.

The DNA-RNA-hybrids resulting from the reverse transcription reaction can also be separated into single strands by means of heat. However, different from heat treatment, the use of RNases has the further advantage that genomic DNA present in the sample is not converted to single stranded form, hence it will not act as a hybridisation template for the primers used in the following steps of the procedure. Special advantages result from the use of RNase I, because this enzyme can easily be inactivated at temperatures below those resulting in denaturation of the genomic DNA. The aim of the process according to the invention is the amplification of ribonucleic acids, hence the use of a stable RNase could hinder this process and would necessitate elimination by elaborate procedures.

In step (c) a single stranded primer is used, which comprises a promoter sequence. A promoter sequence allows the binding of the RNA polymerase and initiates the synthesis of an RNA strand. The use of a single stranded primer comprising the sequence of a highly specific RNA-polymerase promoter like T7, T3 or SP6 is preferred in (c).

The primer is preferably not longer than 35 nucleotides, however a length of not more than 30 nucleotides is especially preferred. The choice of primers of optimal length is of major importance for the process according to the invention. Because other known methods frequently apply too long primers (up to 60 and more nucleotides) which are prone to self-hybridise and lead to vast amounts of artefacts.

According to one embodiment of the process according to the invention a primer is used in step (c) that comprises a promoter sequence and in addition a sequence of maximally 6, preferably only 3 randomly chosen nucleotides. These additional nucleotides allow even hybridisation with any DNA sequence, thus resulting in even amplification of all DNA sequences in the starting population.

The process according to the invention showed especially good results if a primer was used which comprises further to the promoter a sequence of 6 nucleotides, namely the sequence: 5‘-N—N—N-T-C-T-’3, wherein N is any of the following nucleotides: dA, dC, dG, or dT.

In an especially preferred embodiment of the process according to the invention, the single stranded primer used in step (c) can have a length of 27 nucleotides with the following sequence (SEQ ID NO: 1 in the sequence protocol):

5′-A-C-T-A-A-T-A-C-G-A-C-T-C-A-C-T-A-T-A-G-G-N-N-N-T-C-T-3′

The letter N used in SEQ ID NO: 1 represents any of the following nucleotides: dA, dC, dG, or dT. The primer comprises the sequence of the T7-RNA-polymerase-promoter. The transcription start of T7-RNA-polymerase is indicated by⁺¹ in the sequence shown above which is only partially repeated here: 5′-T-A-T-A-G⁺¹-G-N—N—N-3′.

The primer with the above mentioned sequence SEQ ID NO: 1 is also an embodiment of the present invention.

In steps (c) and (e), any DNA-dependent DNA-polymerase can be used. Preferably the Klenow-fragment of the DNA-polymerase is used. It is especially advantageous in the process according to the invention to use the Klenow-exo DNA-polymerase. For the DNA polymerisation in steps (a), (c) and (e) also deoxyribonucleotide monomers are needed, usually dATP, dCTP, dGTP and dTTP.

In step (d), separation of double stranded DNA into single strands can be achieved by any procedure. However, this is preferably done by means of heat.

The single stranded primer used in step (e) can have the identical sequence as the primer used in step (c) or can have a different sequence. However, in the process according to the invention, it is preferred that the primers used in steps (c) and (e) have the same sequence.

Before proceeding to step (f) it may be advisable to remove excess of primers and/or primer induced artefacts (e.g., primer dimers).

The specific RNA-polymerase in step (f) depends on the promoter sequence used in the primer sequence. If the primer comprises a T7-polymerase sequence, then a T7-RNA-polymerase has to be used in step (f).

To obtain ribonucleic acids in step (f), also ribonucleotide-monomers are needed, usually ATP, CTP GTP and UTP.

For the first time, the process according to the invention allows a strong and specific amplification of the starting RNA sequences, representing the total sequences of the entire RNA population. The amplification factor of the starting RNA sequence is at least 500, whereas a factor 3000 is especially preferred.

Specific advantages are obtained using a process as described above, wherein in step
(a) a 5′-(dT)₁₈V-primer is used for reverse transcription; and in
(b) an RNase is used to digest the DNA-RNA-hybrids; and in
(c) and (e) a primer having the SEQ ID NO: 1 and the Klenow-exo DNA polymerase are used; and in
(a) strand separation of double stranded nucleic acids is obtained using heat; and in
(b) excess primers and/or primer-induced artefacts are first removed and then T7-RNA-polymerase is used.

Further amplification of ribonucleic acids can be achieved if the double stranded DNA obtained in step (e) is amplified by at least one PCR cycle. For this purpose the double stranded DNA obtained in step (e) has to be separated into single strands. Using at least one single stranded primer, a DNA polymerase and the deoxyribonucleotide-monomers, DNA strands complementary to the original single stranded DNAs will be produced. Separation of double stranded DNA is achieved preferentially by means of heat. Further amplification of the ribonucleic acids can be achieved if more PCR cycles are performed, preferably using at least 2 or 5 PCR cycles.

This procedure has the special advantages that during the subsequent RNA polymerisation, RNA molecules of both orientations (the original, as well as the complementary sequence) will be obtained.

Further advantages are obtained if during the PCR reaction single stranded primers are used with the same sequence as those used in steps (c) and/or (e). Particularly preferred is the use of single stranded primers of SEQ ID NO: 1.

Also in this version of the process according to the invention, it may be advantageous to remove excess primers and primer induced artefacts (e.g., primer-dimers) before adding the RNA polymerase.

The present invention furthermore relates to kits which comprises all reagents needed to amplify ribonucleic acids by means of the process according to the invention. Respective kits comprise the following components:
(a) at least one single stranded primer comprising a promoter sequence;
(b) an RNA-dependent DNA polymerase;
(c) deoxyribonucleotide-monomers;
(d) a DNA-dependent DNA polymerase;
(e) an RNA polymerase; and
(f) ribonucleotide monomers.

The kit can comprise two or more different single stranded primers. Preferably, one of these primers comprises an oligo-dT-sequence. In one special variant of the process according to the invention, the kit comprises one primer including a 5′-(dT)₁₈V-primer sequence, with V being any deoxyribonucleotide different from dT.

In addition, the kit may comprise RNase I and/or RNase H and/or a single stranded primer, comprising a T7-, T3- or SP6-RNA-polymerase promoter. In addition to the promoter, this primer comprises a random sequence of maximally 6 nucleotides. In particular, the primer can have the SEQ ID NO: 1.

The kit further comprises a DNA-polymerase, preferably the Klenow-fragment of the DNA polymerase and especially preferred is the Klenow-exo DNA-polymerase.

Finally, the kit may comprise a T7-RNA polymerase, a mixture of reagents necessary to label or detect RNA and/or DNA and further include one or several microarrays. Herewith, the kit may comprise all components needed to conduct gene expression analysis.

According to the invention it is especially preferred that the kit comprises the following components:
(a) a 5′-(dT)18V-primer for reverse transcription;
(b) RNase;
(c) a primer having the sequence shown in SEQ ID NO: 1;
(d) Klenow-exo DNA-polymerase;
(e) T7-RNA polymerase.

The different components of the kit will normally be supplied in different tubes. However, it is possible that components used in the same step of the procedure will be supplied in one tube.

Therefore, the present invention also relates to processes for the analysis of nucleic acids, wherein ribonucleic acids are obtained and amplified using any of the procedures described in the present invention and which will thereafter be analyzed using a microarray technique. Ribonucleic acids are normally isolated from biological samples. Prior to microarray analysis, ribonucleic acids amplified by techniques of the present invention may be transcribed into cDNA, using a reverse transcription. The present invention allows analysis of the amount and/or sequence of the cDNA.

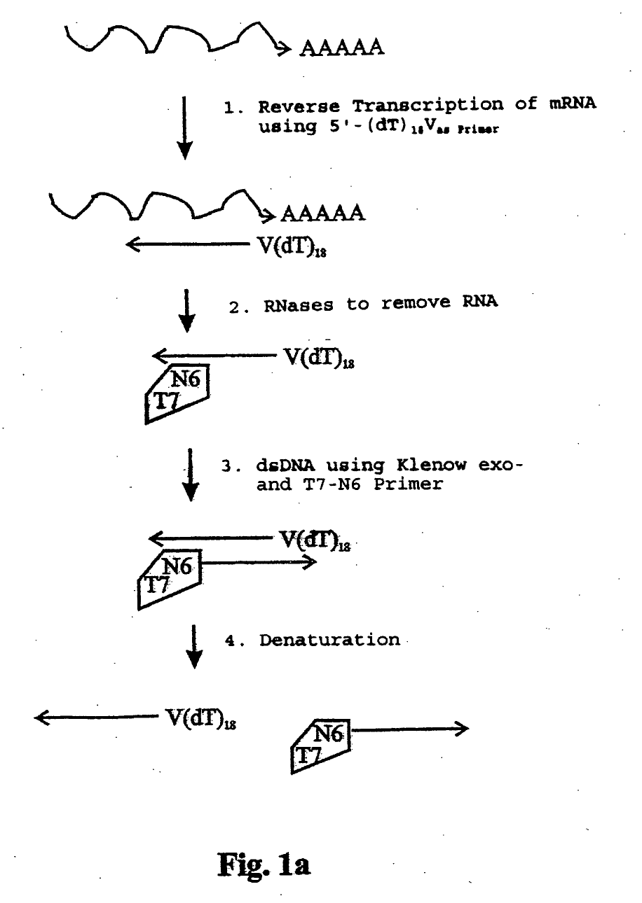

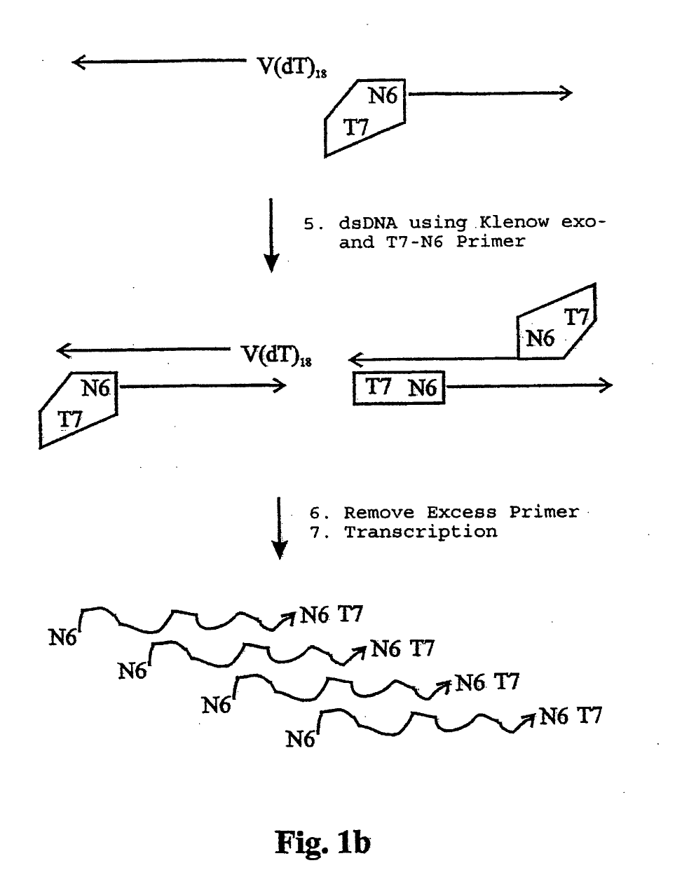

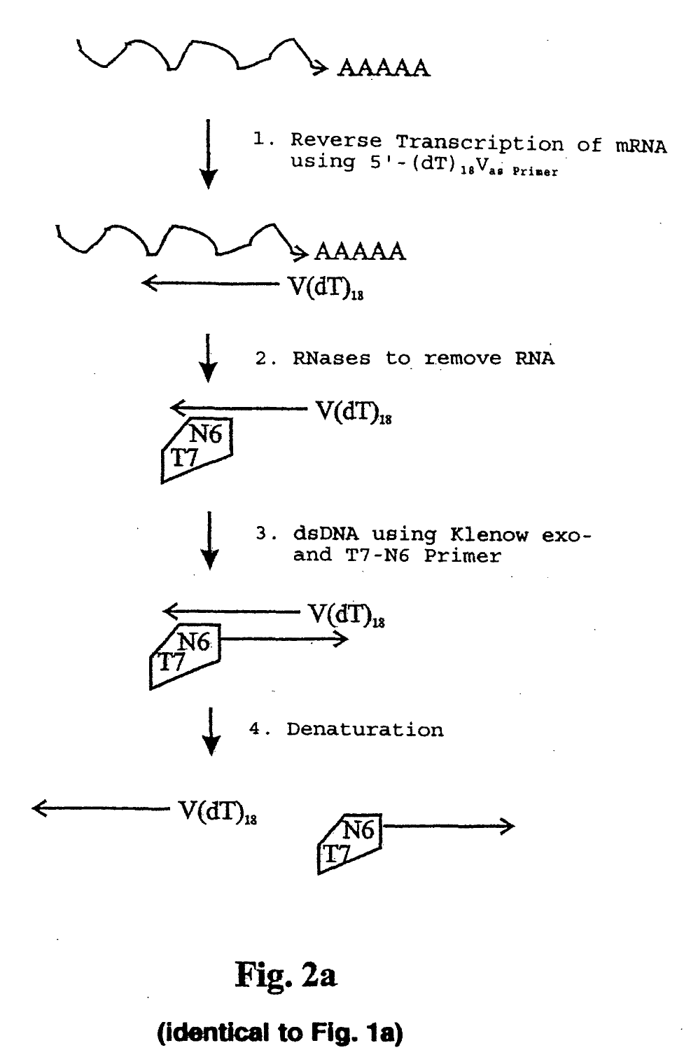

FIG. 1 shows a schematic diagram of the processes of the present invention: In a first step RNA is transcribed into single stranded DNA by means of reverse transcription, using an anchored oligo(dT)₁₈V primer. This procedure allows the reverse transcription starting at the ploy-A tail of the mRNA to the 3′-UTR area. The next step eliminates the RNA from the RNA-cDNA-heteroduplex by use of RNase H and the residual RNA (ribosomal RNA) is digested by RNase I.

Synthesis of the second, complementary DNA strand is used to introduce the T7-promoter sequence via a special primer. This primer consists in one part of 6 random nucleotides and a second part which comprises the T7-polymerase promoter sequence. Alternatively, the primer having the sequence of SEQ ID NO: 1 can be used.

After primer annealing, elongation to double stranded DNA is achieved by incubation with the Klenow-fragment of the DNA polymerase. Heat-induced denaturation of the DNA double strand is followed by a reduction of the incubation temperature, so that the primer of the present invention can again hybridise with the DNA. A further DNA strand is obtained by primer elongation. Subsequently excess primer and primer-induced artefacts (primer dimers) are removed and the RNA amplification is achieved by in vitro transcription using the T7 promoter.

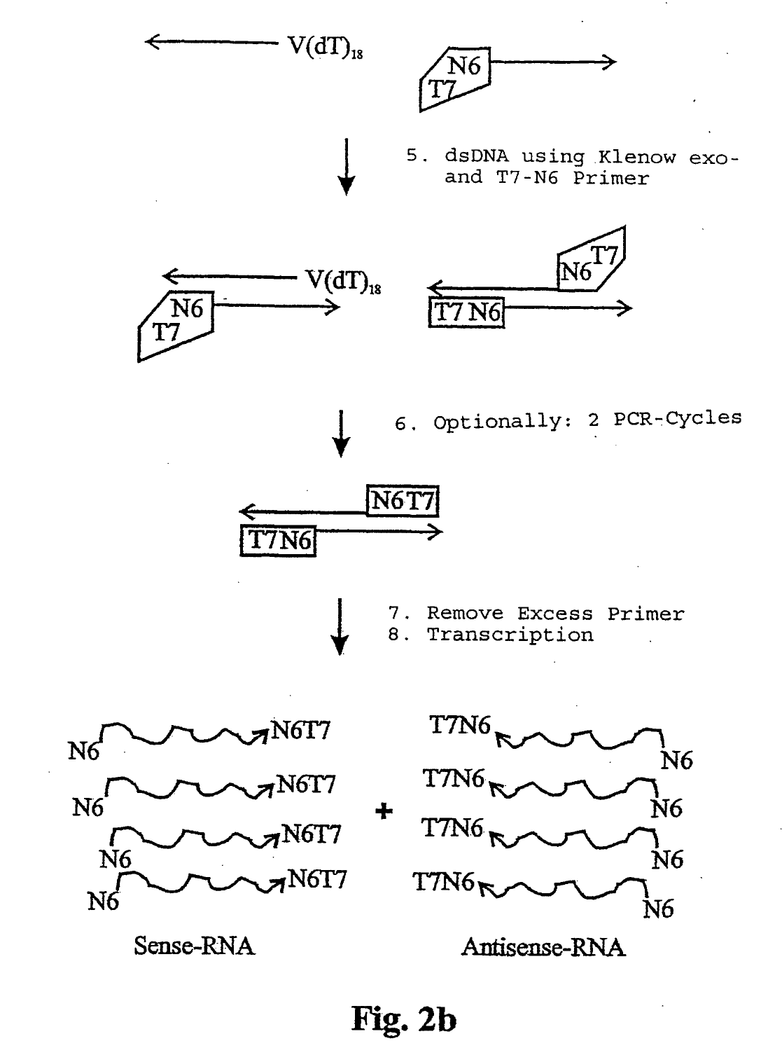

An alternative to the above process is shown in FIG. 2. This alternative procedure includes amplification of the double stranded DNA, by means of PCR, prior to the transcription reaction. As shown in FIG. 2, this alternative procedure allows the production of ribonucleic acids with identical sequence as the starting material as well as the production of ribonucleic acids with the complementary sequence.

The order and detailed implementation of the reaction steps of the present invention are shown by way of examples:

### EXAMPLES

### Example 1

### Reverse Transcription of 100 ng Total-RNA Using Oligo(dT)₁₈V-Primer

| First strand-DNA-Synthesis: | |
|---|---|
| RNA (50 ng/μl): | 2 μl |
| Oligo(dT)₁₈ V(5 pmol/μl): | 1 μl |
| dNTP-Mix (10 mM): | 0.5 μl |
| DEPC-H₂O | 2 μl |

Incubate 4 min at 65° C. in a thermocycler with a heated lid, then place immediately on ice.

| Mastermix for synthesis of the 1^{st} strand of cDNA | |
|---|---|
| 5 × RT-buffer | 2 μl |
| 100 mM DTT | 1 μl |
| RNase-inhibitor (20 U/μl) | 1 μl |
| Superscript II (200 U/μl) | 0.5 μl |

Pipette components for the mastermix on ice and add to the tube containing the reverse transcription mix. Place samples in a thermocycler (preheated to 42° C.)

Incubate as follows:

42° C./50 minutes
45° C./10 minutes
50° C./10 minutes
70° C./15 minutes (enzyme inactivation)
   Place samples on ice.

### Example 2

### RNA Elimination

| Elimination of RNA from the reaction | |
|---|---|
| First strand-cDNA mix | 10 μl |
| RNase-Mix (RNase H/RNase I; each at 5 U/μl) | 1 μl |

Incubate for 20 min at 37° C., hereafter place samples on ice. RNase A was not used for RNA elimination. Because RNase A is not readily inactivated. RNase I on the other hand, the enzyme used in this invention, can be inactivated easily and completely by incubation at 70° C. for 15 min.

### Example 3

### Random Forward- and Reverse-Priming of First Strand Cdna with T7-Random-Primer

| Random priming of first strand cDNA with T7-random Primer | |
|---|---|
| First strand-cDNA | 10 μl |
| dNTP-mix (10 mM) | 0.5 μl |
| artus 6 (T7-random-Primer, 10 pmol/μl) | 3 μl |
| 10 × Klenow buffer | 5 μl |
| H₂O | 30.5 μl |

Incubation:
Forward-priming:
65° C./1 minute
37° C./2 minutes
add 1 μl Klenow-exo⁻ (5 U/μl) to each sample
incubate at 37° C./20 minutes
   Reverse-priming:
95° C./1 minute
37° C./2 minutes
add 1 μl Klenow-exo⁻ (5 U/μl) to each sample
incubate at 37° C./20 minutes
65° C./15 minutes (enzyme inactivation)

### Example 4

### Purification of the cDNA with High-Pure PCR Purification Kit (Roche)

| cDNA purification | |
|---|---|
| Klenow-Reaction mix | 50 μl |
| Binding-buffer | 250 μl |
| Carrier (cot-1-DNA, 100 ng/μl) | 3 μl |

Transfer mix onto provided columns, spin in a tabletop centrifuge at maximal rpm for 1 min. Discard the flow-through. Add 500 μl washing buffer to the column and spin as above, discard flow-through and repeat the wash step with 200 μl washing buffer. Transfer columns onto a new 1.5 ml reaction tube add 50 μl elution buffer, incubate for 1 min at RT and centrifuge as described above. Repeat the elution step once, again using 5011 buffer.

### Example 5

### Ethanol-Precipitation of Purified cDNA

Do not vortex the Pellet Paint™-carrier stock solution and store in the dark. Keep at −20° C. for long term storage, smaller aliquots can be stored for approximately 1 month at 4° C.

| Ethanol-precipitation | |
|---|---|
| Elute | 100 μl |
| Carrier (Pellet Paint ™) | 2 μl |
| Sodium-acetate | 10 μl |
| Ethanol; absolute | 220 μl |

Mix thoroughly (do not vortex) and pellet cDNA by centrifugation at maximal rpm for 10 min at RT. Discard supernatant; wash pellet once with 200 μl 70% ethanol. Centrifuge for 1 min as described above. Remove supernatant completely using a pipette. Dry pellet by incubation of the open reaction tube for 5 min at RT. The samples should not be dried in a speed vacuum! Dissolve pellet in 8 μl Tris-buffer (pH 8.5) and place on ice.

### Example 6

### Amplification by in vitro-Transcription

| In vitro transcription | |
|---|---|
| CDNA | 8 μl |
| UTP (75 mM) | 2 μl |
| ATP (75 mM) | 2 μl |
| CTP (75 mM) | 2 μl |
| GTP (75 mM) | 2 μl |
| 10 × buffer | 2 μl |
| T7-RNA-Polymerase | 2 μl |

Thaw all components and mix them at RT, and not on ice, because the spermidine component of the reaction buffer would induce precipitation of the template. Use 0.5 ml or 0.2 ml RNase-free PCR tubes for this step.

Incubate the transcription reaction overnight at 37° C. either in a thermocycler with heated lid (at 37° C.) or in a hybridisation oven. Load 1-2 μl of the reaction mix onto a 1.5% native agarose gel. Add 1 μl DNase to the remaining reaction and incubate for further 15 min at 37° C. To purify the RNA use the RNeasy kit from Qiagen according to the manufacture's protocol for RNA-clean-up. At the end of the cleanup procedure, elute the RNA by using 2×50 μl DEPC-water and perform an ethanol precipitation as described above in step 6. Dissolve RNA pellet in 5 μl DEPC water.

The RNA is now ready for labelling and use in a microarray hybridisation.

1. A method for the amplification of ribonucleic acids comprising
(a) reverse transcribing a single stranded DNA from an RNA template, using a first single stranded primer, an RNA-dependent DNA polymerase and deoxyribonucleotide monomers;
(b) removing the RNA template;
(c) synthesizing a first double stranded DNA, using a second single stranded primer comprising a promoter sequence, a DNA polymerase and deoxyribonucleotide monomers;
(d) separating the first double stranded DNA into single stranded DNAs;
(e) synthesizing a second double stranded DNA from a single stranded DNA obtained in (d), using a third single stranded primer comprising a promoter sequence, a DNA polymerase and deoxyribonucleotide monomers;
(f) synthesizing multiple single stranded RNAs, using an RNA polymerase and ribonucleotide monomers.

2. The method according to claim 1, wherein the single stranded RNAs have the same sense orientation as the RNA template.

3. The method according to claim 1, wherein the first single stranded primer comprises an oligo-dT-sequence.

4. The method according to claim 1, wherein the first single stranded primer is a 5′-(dT)₁₈V-primer, and wherein V is any deoxyribonucleotide monomer different from dT.

5. The method according to claim 1, wherein the RNA template is removed using RNase in step (b).

6. The method according to claim 1, wherein the RNA template is removed using RNase or RNase H in step (b).

7. The method according to claim 1, wherein the RNA template is removed using RNase I and RNase H in step (b).

8. The method according to claim 1, wherein the second single stranded primer comprises the sequence of a T7-, T3-, or SP6—RNA-polymerase promoter.

9. The method according to claim 1, wherein the second single stranded primer further comprises a random sequence of not more than 6 nucleotides.

10. The method according to claim 1, wherein the second single stranded primer has a total length of not more than 35 nucleotides.

11. The method according to claim 1, wherein the second single stranded primer has a total length of not more than 30 nucleotides.

12. The method according to claim 1, wherein the second single stranded primer comprises a sequence of SEQ ID NO: 1.

13. The method according to claim 1, wherein the DNA polymerase is Klenow-fragment DNA polymerase.

14. The method according to claim 1, wherein the DNA polymerase is Klenow-exo DNA polymerase.

15. The method according to claim 1, wherein the deoxyribonucleotide monomers are dATP, dCTP, dGTP and dTTP

16. The method according to claim 1, wherein the separating the first double stranded DNA into single stranded DNAs is accomplished by heating.

17. The method according to claim 1, wherein the second single stranded primer is the same as the third single stranded primer.

18. The method according to claim 1, wherein the second single stranded primer is different from the third single stranded primer.

19. The method according to claim 1, wherein the RNA polymerase is T7-RNA polymerase.

20. The method according to claim 19, wherein excess primer and primer-induced artefacts are removed prior to incubation with T7-RNA-polymerase.

21. The method according to claim 19, wherein excess primer or primer-induced artefacts are removed prior to incubation with T7-RNA-polymerase.

22. The method according to claim 1, wherein ATP, CTP, GTP and UTP are used as ribonucleotide monomers.

23. The method according to claim 1, wherein the amplification factor of the RNA template is at least 500.

24. The method according to claim 1, wherein the amplification factor of the RNA template is at least 3000.

25. The method according to claim 1, wherein in step
(a) a 5′-(dT)18V-primer is used for reverse transcription; and in
(b) an RNase is used to remove the RNA template; and in
(c) and (e) a primer comprising SEQ ID NO: 1 and the Klenow-exo DNA polymerase are used; and in
(d) separating the first double stranded DNA into single stranded DNAs is accomplished by heating; and in
(e) excess primers and primer-induced artefacts are first removed and then T7-RNA-polymerase is used.

26. The method according to claim 1, wherein the second double stranded DNA is separated into single strands and complementary DNA strands to each single strand are prepared using at least one single stranded primer, a DNA polymerase and the deoxyribonucleotide monomers.

27. The method according to claim 26, wherein strand separation, primer annealing and elongation are repeated at least 2 or 5 times.

28. The method according to claim 26, wherein strand separation, primer annealing and elongation are repeated at least once.

29. The method according to claim 26, wherein strand separation is accomplished by heating.

30. The method according to claim 26, wherein further DNA double strands are produced using single stranded primers with the same sequence as the primer used in (c) and/or (e).

31. The method according to claim 26, wherein the second single stranded primer comprises a sequence of SEQ ID NO: 1.

32. The method according to claim 26, wherein the second single stranded primer is the same as the third single stranded primer.

33. The method according to claim 26, wherein ribonucleic acids are produced with the same sense orientation as the starting material and simultaneously also with the complementary sequence orientation.

34. A kit for the amplification of ribonucleic acids according to claim 1, which comprises the following components:
(g) at least one single stranded primer comprising a promoter sequence;
(h) an RNA-dependent DNA polymerase;
(i) deoxyribonucleotide monomers;
(j) a DNA-dependent DNA polymerase;
(k) an RNA polymerase; and
(l) ribonucleotide monomers.

35. The kit according to claim 34, wherein the kit comprises two different single stranded primers.

36. The kit according to claim 35, wherein one single stranded primer comprises an oligo-dT-sequence.

37. The kit according to claim 34, wherein a single stranded primer comprises a 5′-(dT)₁₈V-primer sequence for reverse transcription, with V being any deoxyribonucleotide-monomer different from dT.

38. The kit according to claim 34, further comprising RNase I and/or RNase H.

39. The kit according to claim 34, comprising a single stranded primer with a T7, T3 or SP6 RNA-polymerase promoter sequence.

40. The kit according to claim 34, wherein the at least one single stranded primer further comprises a random sequence of not more than 6 nucleotides.

41. The kit according to claim 34, comprising a single stranded primer comprising a sequence of SEQ ID NO: 1.

42. The kit according to claim 34, comprising the Klenow-fragment of the DNA polymerase.

43. The kit according to claim 34, comprising the Klenow-exo DNA polymerase.

44. The kit according to claim 34, comprising the T7-RNA polymerase.

45. The kit according to claim 34, comprising a set of reagents for labelling and detection of nucleic acids.

46. The kit according to claim 34, further comprising:
(a) a 5′-(dT)₁₈V-primer for reverse transcription;
(b) RNase;
(c) a primer comprising a sequence of SEQ ID NO: 1;
(d) Klenow-exo DNA-polymerase; and
(e) T7-RNA polymerase.

47. The kit according to claim 34 comprising a microarray.

48. The kit according to claim 46 comprising a microarray.

49. A method for the analysis of nucleic acids, wherein ribonucleic acids are obtained, amplified using a process according to claim 1 and analyzed using a microarray.

50. A method according to claim 49, wherein the ribonucleic acids are obtained from a biological sample.

51. A method according to claim 49, wherein the ribonucleic acids are amplified, converted to cDNA by means of reverse transcription, and the cDNAs are analyzed by uising micoarrays.

52. A method according to claim 49, wherein the amount and sequence of the cDNA are analyzed.

53. A method according to claim 49, wherein the amount or sequence of the cDNA are analyzed.

54. A nucleic acid comprising a sequence of SEQ ID NO: 1.
